(19) United States
(12) Patent Application Publication
(10) Pub. No.: US 2024/0335634 A1
(43) Pub. Date: Oct. 10, 2024

(54) Headset for Neural Conditioning Based on Plural Feedback Signals

(71) Applicant: Vital Neuro, Inc., New York, NY (US)

(72) Inventors: Kamran Fallahpour, New York, NY (US); John Golden, New York, NY (US)

(21) Appl. No.: 18/612,113

(22) Filed: Mar. 21, 2024

Related U.S. Application Data
(60) Provisional application No. 63/457,949, filed on Apr. 7, 2023.

(51) Int. Cl. A61M 21/00

(52) U.S. Cl. CPC A61M 21/00; A61M 2021/0027; A61M 2021/005; A61M 2210/0693; A61M 2230/10

(57) ABSTRACT

A brain-training system for training neurons in a human's head to transition into a desired state includes a headset that comprises an electroencephalograph that extends along an arcuate path in a median plane of the human's head. The electroencephalograph comprises plural dry electrodes. The brain-training system further comprises an additional electrode that is configured to measure an additional physiological signal.

### CROSS REFERENCE TO RELATED APPLICATIONS

This application claims priority to U.S. Application No. 63/457,949, filed Apr. 7, 2023, the contents of which is hereby incorporated by reference in its entirety.

### BACKGROUND

A human brain comprises neurons that engage in electrical activity. This electrical activity can be detected non-invasively by placing electrodes on the scalp. The resulting waveforms, referred to colloquially as “brain waves,” provide some insight into the human's mind. Changes in these brain waves thus provide a way to detect a state change in the human's mind.

For example, certain types of brain waves are known to be associated with sleep or extreme relaxation. Disturbances in these brain waves thus imply that a subject may have become aroused. Other types of brain waves are known, from observation, to be indicative of a state of attention. Changes in such brain waves thus provide a basis for inferring that a subject may have lapsed into a state of distraction.

A human subject has some control over such state changes and may attempt to essentially will oneself to focus or to lull oneself into a state of relaxation.

However, a human's ability to control state-of-mind is limited and also varies widely across the population. There are many people whose ability to transition into and remain in a state of attention is quite limited. In some cases, this inability is sufficiently extreme such that pharmaceuticals are used to artificially induce the desired state. There are also large swaths of the population that have difficulty relaxing. Again, such people often resort to pharmaceuticals in order to reach the desired state of consciousness.

### SUMMARY

In one aspect, the invention features a brain-training system for training neurons in a human's head to transition into a desired state. The brain-training system includes a headset that includes an electroencephalograph that extends along an arcuate path in a median plane of the human's head. The electroencephalograph includes plural dry electrodes. In addition, the brain-training system includes an additional electrode that is configured to measure an additional physiological signal.

Embodiments include those in which the additional electrode is configured for detecting an electromyographic signal, those in which one of the dry electrodes also functions as the additional electrode, those in which the additional electrode is a dry electrode, those in which the additional electrode is a wet electrode, those in which additional electrode is disposed on the human's frontalis muscle, and those in which one of the dry electrodes is used as the additional electrode.

Still other embodiments include those in which the additional electrode is a dry electrode that is extended to over a suitable muscle of interest, such as the human's frontalis muscle or the human's orbicularis oculi.

Also among the embodiments are those in in which the headset includes a reference sensor on an earcup thereof and the reference sensor is used as the additional electrode and those.

Yet other embodiments include those in which the headset includes an earcup and the additional electrode is on the earcup.

In another aspect, the invention features method that comprises exposing a subject to a test protocol while obtaining real-time electroencephalographic measurements and real-time measurements of an additional physiological signal and, based on the real-time measurements, determining a likelihood that the subject suffers from a disorder.

In some practices of the method, the disorder is PTSD.

Practices also include those in which the additional physiological signal is an electromyographic signal, those in which the additional physiological signal is indicative of activity by the subject's orbicularis oculi, and those in which it is indicative of activity by the subject's frontalis.

Still other practices include those in which the test protocol includes plural audio segments separated by rests, wherein each of the audio segments includes instances of a tone, the instances having different volumes and different durations, those in which the test protocol includes an audio segment, those in which the test protocol includes a video segment, and those in which the test protocol includes both an audio segment and a video segment.

Practices further includes those in which the real-time measurements are indicative of the subject's response to the test protocol and those in which the real-time measurements are indicative of the subject's startle response to the test protocol.

Still other practices include those in which the test protocol includes a stimulus that is not consciously perceptible by the subject, those in which the test protocol includes an audio stimulus that is not consciously perceptible by the subject, those in which the test protocol includes a video stimulus that is not consciously perceptible by the subject, and those in which the test protocol includes both an audio segment and a video segment, neither of which are consciously perceptible by the subject.

Further practices include those in which additional physiological signal is an electromyographic signal, those in which the additional physiological signal is a signal indicative of heart rate of the subject, those in which the additional physiological signal is a signal indicative of heart rate variability of the subject, and those in which the additional physiological signal is a signal indicative of a galvanic skin response of the subject.

These and other features of the invention will be apparent from the following detailed description and the accompanying figures, in which:

### DESCRIPTION OF DRAWINGS

FIG. 1 shows a brain-training system comprising a headset;

FIG. 2 shows details of the headset shown in FIG. 1;

FIG. 3 shows circuitry in the headset shown in FIG. 1;

FIG. 4 shows constituents of a stimulus generated by the brain-training system of FIG. 1;

FIG. 5 shows the fourth electrode of FIG. 2 after having been placed to detect an electromyographic signal from a muscle under a subject's eye;

FIG. 6 shows a protocol having audio segments separated by rests for use in stimulating the subject shown in FIG. 5; and

FIG. 7 shows details of an audio segment from FIG. 6.

### DETAILED DESCRIPTION

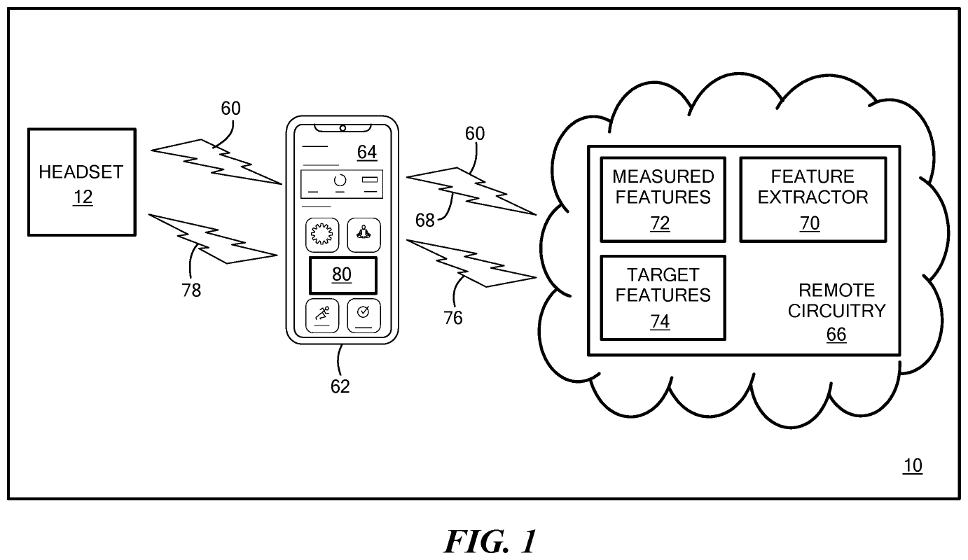

FIG. 1 shows a brain-training system 10 for conditioning neurons in the brain of a subject. The brain-training system 10 conditions the neurons into cooperating to cause a transition into a desired mental state and to thereafter cause the neurons to remain in that mental state. Examples of mental states include a relaxed state, a stress-reducing state, a state conducive to sleep, a state conducive to enhancement of focus, a state that relieves symptoms of such conditions as post-traumatic stress disorder (“PTSD”), and a state conducive to greater attention.

For ease of discussion, it will be useful to define certain directions relative to a subject's head. Accordingly, as used herein, a “median plane” is a plane that bisects the head along the corpus callosum into left and right hemispheres. As used herein, a “transverse plane” is a plane that is perpendicular to the medial plane and passes through the ears. The “coronal plane” is one that is perpendicular to the transverse and median planes. A “path” in any of the foregoing planes is a continuous set of points in that plane having two endpoints.

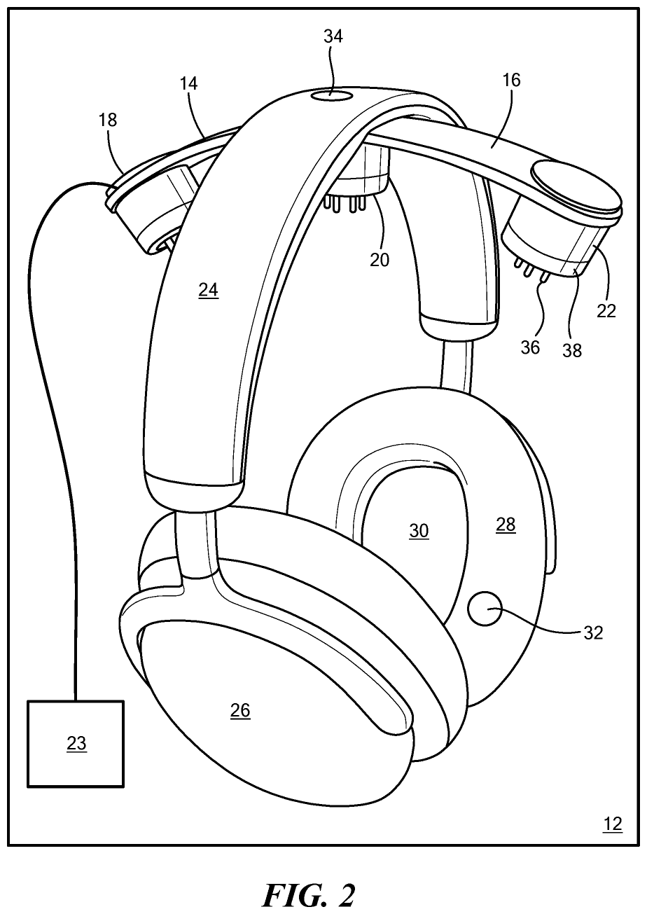

The training system 10 includes a headset 12. As shown in FIG. 2, the headset 12 comprises an electroencephalograph 14. The electroencephalograph 14 includes a housing 16 that extends along an arcuate path in the median plane so as to roughly follow the contour of the head along a medial direction. The housing 16 supports first, second, and third electrodes 18, 20, 22. These are dry electrodes that do not require a conductive gel to make contact. In some embodiments, the housing is rigid. However, in others, the housing is flexible and thus adjustable to more closely follow the contour of the head along the medial direction.

The training system 10 further includes a fourth electrode 23 that can be used to sense an additional physiological signal. Examples of other physiological signals include temperature, heart rate, heart-rate variability, glucose level, blood pressure, signals indicative of hydration, and signals indicative of activity or movement, including electromyographic (EMG) signals.

In FIG. 2, the fourth electrode 23 is shown connected to the headset 12. However, in some embodiments, one or more of the first, second, and third electrodes 18, 20, 22 doubles as the fourth electrode 23. In such cases, the signal received by that doubling electrode is a superposition of the additional physiological signal and the signal that that electrode 18, 20, 22 would otherwise have received. Such embodiments rely on additional signal processing steps to separate out the additional physiological signal.

In a preferred embodiment, the headset 12 also comprises a headband 24 that stabilizes the electroencephalograph 14. The headband 24 extends along an arcuate path in the coronal plane so as to follow the contour of the head along the direction in which the coronal plane extends. In addition to its role as a stabilizer for the electroencephalograph 14, the headband 24 also supports first and second earcups 26, 28 at ends thereof. Each earcup 26, 28 comprises a loudspeaker 30 that is to be used in connection with neural conditioning. At least one earcup comprises a ground contact 32 to provide a reference voltage for the electrodes 18, 20, 22.

In some embodiments, a fastener 34 provides a mechanical coupling between the housing 16 and the headband 24. As a result, the headband 24 can be separated from the headset 12 and later reattached to the headset 12. In other embodiments, the housing 16 is integral with the headset 12 and therefore cannot be detached from the headset 12. In either case, the headband 24 and the housing 16 are positioned relative to each other such that when a subject 25 wears the headset 12, each of the first, second, and third electrodes 18, 20, 22 makes contact with a corresponding site of the subject's scalp so as to provide real-time monitoring of brain waves emanating from that site.

The sites are selected based on the mental state that the subject's neurons are to be trained to achieve. In particular, the sites are chosen to be adjacent to the locations of neurons that are known to be pertinent to assessing the existence of a selected mental state. The three sites shown in FIG. 3 are the posterior cortex, the anterior cortex, and the motor/sensory cortex. Such placement is useful for conditioning the brain to transition into states of greater attention, focus, and relaxation, respectively. Suitable placements, using the International 10-20 system, are Fz, Cz, and Pz.

The earcups 26, 28 serve as fiducials for correct placement of the first, second, and third electrodes 18, 20, 22. In particular, the housing 16 is disposed on the headset 12 such that when the earcups 26, 28 are in the correct position over the subject's ears, the first, second, and third electrodes 18, 20, 22 will be disposed over the correct sites on the subject's scalp. In the illustrated embodiment, the first, second, and third electrodes 18, 20, 22 are placed relative to each other so that if one is disposed over the first site, the other two are disposed over the second and third sites respectively. Each of the first, second, and third electrode 18, 20, 22 receives brain waves from the corresponding sites over which it is disposed. In general, it is not necessary to activate all three electrodes.

Because hair is often found on the scalp, it is useful for each of the first, second, and third electrodes 18, 20, 22 to comprise pins 36 that extend towards the subject's scalp, as shown in FIG. 2. In a preferred embodiment, the pins 36 are biased to project towards the scalp. In such cases, each pin 36 resists a force that tends to reduce the extent to which it projects towards the scalp. Among these embodiments are those in which the pins 36 are spring-loaded pins 36. To provide a more comfortable fit, it is also useful for the pins 36 to protrude from a flexible layer 38. In a typical embodiment, the flexible layer 38 comprises silicone.

Referring now to FIG. 3, the brain-training system 10 features circuitry 40 that comprises a multiplexer 42 for receiving analog signals 44 from the first, second, third, and fourth electrodes 18, 20, 22, 23 The multiplexer 42 also includes a selection input 46 for selecting which of the analog signals 44 is to be passed to an analog-to-digital converter 48. Embodiments include those in which the selected signal is that of only one electrode 18, 20, 22, 23 However, in some embodiments, the multiplexer 42 combines weighted outputs from two or more electrodes 18, 20, 22, 23 thereby effectively creating a synthetic electrode.

The analog-to-digital converter 48 samples the analog signal 44 and quantizes it into discrete levels to form a corresponding digital signal 50. The resulting digital signal 50 is then provided to noise-reduction circuitry 52 and filtering circuitry 54 before being provided to a wireless interface 56 for transmission via an antenna 58 as a measurement signal 60 that is ultimately received by a portable device 62. An example of a portable device 62 is a smartphone, a tablet, smart jewelry, such as a smart watch, or a personal computer.

A training application 64 executing on the portable device 62 will have received, from the subject 25, instructions indicative of what mental state the training system 10 should attempt to achieve.

The wireless interface 56 receives a selection signal 64 from the portable device 62. The selection signal 64 selects the appropriate electrode for use in real-time monitoring. The circuitry 40 causes this selection signal 64 to be provided to the selection input 46 of the multiplexer 42.

Referring back to FIG. 1, the training application 64 uses the digital signals 18 received from the first, second, third, and fourth electrodes 18, 20, 22, 23 to display physiological signals, including brain waves, in real time on the portable device 62. In addition, the training application 64 causes the portable device 62 to forward the measurement signal 60 to remote circuitry 66 in the cloud together with desired-state information 68 representative of what sort of neural conditioning the subject 25 wishes to achieve.

The remote circuitry 66 includes feature-extraction circuitry 70 that carries out feature extraction on the measurement signal 60 to obtain measured feature-set 72 for the subject 25. Based on the desired-state information 68, the remote circuitry 66 defines a target feature-set 74.

The remote circuitry 66 then formulates a conditioning stimulus 76 to which the subject's neurons are to be exposed to begin the conditioning process. The conditioning stimulus 76 is tailored to cause the features present in the baseline to transition into the target features.

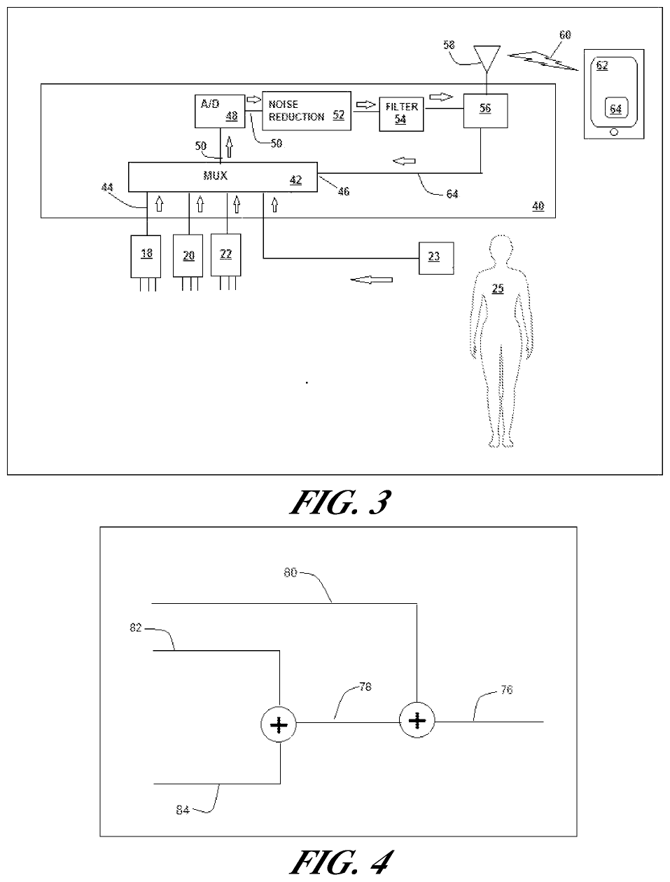

Referring to FIG. 4, the conditioning stimulus 76 comprises an audio constituent 78 and a video constituent 80 to which the subject 25 is to be exposed in an attempt to condition the relevant neurons so that they achieve and maintain the desired brain state.

The remote circuitry 66 transmits the conditioning stimulus 76 to the training application 64, together with synchronizing information to ensure that the audio constituent 78 and video constituent 80 are displayed at the correct times relative to each other. Upon receipt of the conditioning stimulus 76, the training application 64 separates the video constituent 80 from the audio constituent 78 and displays the video constituent 80 on the portable device 62. The training application 64 then sends the audio constituent 78 to the headset 12 to be listened to by the subject 25. As a result, the subject's neurons are exposed to the conditioning stimulus 76 using different sensory pathways.

The training application 64 continues to receive a measurement signals 60 from the electroencephalograph 14 as the subject 25 is exposed to the conditioning stimulus 76. These updated measurement signals 60 are then transmitted to the remote circuitry 66 to serve as a basis for feedback control over the neural conditioning process.

The remote circuitry 66 carries out further feature extraction on the updated measurement signal 60. The resulting updated measured feature-sets 72 provide a basis for evaluating the effect of the conditioning stimulus 76 and, in particular, the progress made towards driving the measured feature-set 72 towards the target feature-set 74. In response to the assessment of such progress, the remote circuitry 66 then formulates a revised conditioning-stimulus 76. It then transmits the revised conditioning-stimulus 76 back to the training application 64 so that the neurons to be conditioned can be exposed to them via the subject's sensory pathways.

The training system 10 thus forms a distributed closed-loop feedback system that attempts to guide the subject's brain waves to achieve a particular feature set through exposure to conditioning stimulus 76, with the conditioning stimulus 76 being adapted periodically in an effort to guide the received feature set towards the target feature set.

In some embodiments, the audio constituent 78 comprises a superposition of a music component 82 and a reward component 84. The reward component 84 is made to appear or disappear or is otherwise altered in response to the progress being made towards arriving at the target feature set. In some embodiments, the reward component 84 is a single tone whereas in others it is a combination of frequencies.

The music component 82 itself can be viewed as a superposition of components. The remote circuitry 66 would therefore be able to also vary the audio constituent 78 of the conditioning stimulus 76 by modifying this superposition of the music's components.

In some cases, the music's components form an orthogonal basis of a function space. For example, the components can be complex exponentials such as those used in a Fourier transform. In such cases, the remote circuitry 66 adaptively varies the conditioning stimulus 76 to suppress or enhance certain frequencies of the music component 82 in an attempt to drive the brain waves to have the desired feature set.

In other cases, the components of the music component 82 do not form an orthogonal basis of the function space. For example, a first component could be the function that, when played by itself, contains the sounds made by the string section and a second component could be the function that, when played by itself, sounds the rest of the orchestra minus the string section from the first component. This granularity of components can be further increased. For example, the components may include a function that contains the sound played by a particular violin.

In either case, the components whose superposition forms the music component 82 can be individually weighted by a complex number so as to modify the amplitude of that component and its phase relative to other components in an attempt to tune the conditioning stimulus 76 to drive the features obtained from the measurement signal 60 towards the target feature. In effect, this generalizes the concept of the reward tone 84 from being restricted to a drone-like sound to a more general transformation of the components of a musical composition.

In still other embodiments, either the audio or video constituent 78, 80 of the conditioning stimulus 76 is adaptively modified based on changes in brain state or in neural activity. Examples include causing the music component 82 to pause, by changing the overall volume of the music component 82 as a whole or on a component-by-component basis, or by changing the perceived source of the audio constituent, for example by varying the relative volumes of the loudspeakers 30.

It should be noted that the act of modifying an existing musical composition by assigning weights to its components can be viewed as effectively creating a new composition. As a result, the remote circuitry 66 can be viewed as adaptively composing a music component 82 in an effort to condition neurons in the subject's brain to achieve a desired state, the desired state having been defined by the target features.

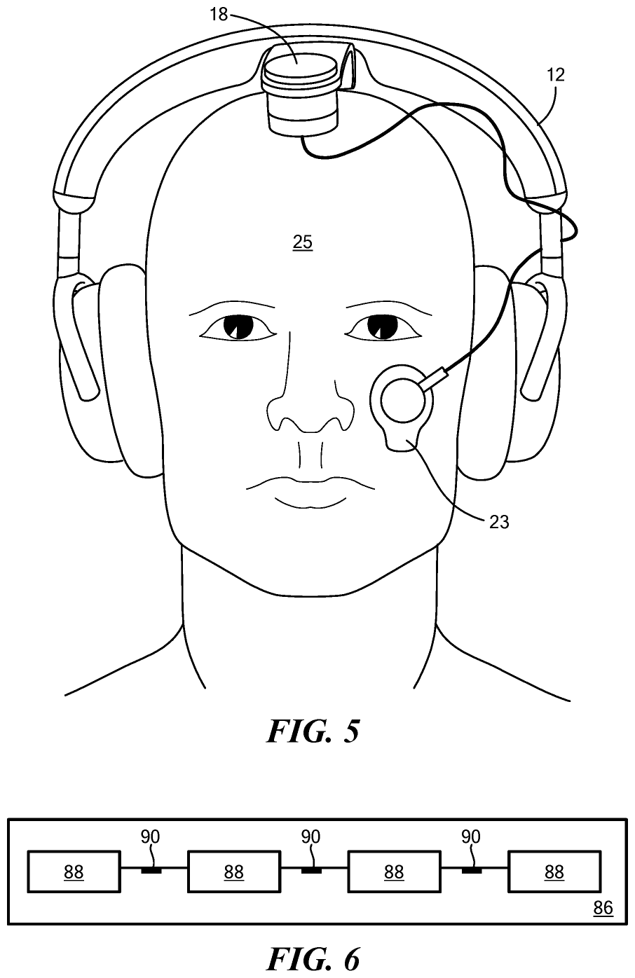

FIG. 5 shows a particular embodiment for use in connection with a diagnostic test for PTSD on the subject 25.

In the illustrated embodiment, the subject 25 wears the headset 12 with its first electrode 18 having been removed and the remaining socket connected to the fourth electrode 23, which is disposed under the subject's eye or directly over the orbicularis oculi. This is a particularly suitable location because, in response to stimulus intended to startle the subject 25, the extent to which the subject's orbicularis oculi twitches is indicative of a likelihood of PTSD. The fourth electrode 23 in this embodiment is a disposable disk electrode. A suitable disk electrode is one that relies on a reaction between silver and chlorine ions to form a silver chloride precipitate.

In some embodiments, an electrode 18, 20, 22 that is normally used to receive an electroencephalographic signal can also be used to measure an electromyographic signal. This can be done by extending it so that it is close enough to a suitable muscle whose electromyographic activity is of interest. A particularly useful one of the electrodes is that positioned over the Fz. In those cases in which the same sensor is used for receiving both an electroencephalographic signal and an electromyographic signal, the output will be a superposition of both signals. In such cases, it is useful to carry out additional signal processing steps, such as filtration or execution of an electromyographic signal detection algorithm in order to separate out the two signals from background noise and from each other.

In the case of observing the PTSD startle response, suitable muscles for observation include the orbicularis oculi and the frontalis. The EMG startle response may also be detected from the Fz EEG sensor directly with finetuning the filtration and EMG detection algorithms.

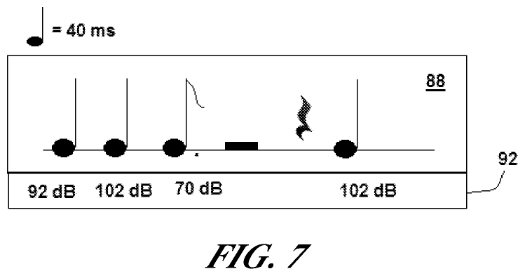

Once the various electrodes 18, 20, 22, 23 are in place, the training application 64 initiates a testing protocol 86, as shown in FIG. 6. A suitable testing protocol 86 features a sequence of audio segments 88 separated by rests 90. The subject 25 hears the testing protocol 86 using the loudspeaker 30 on the headset 12. In some embodiments, these audio segments 88 are integrated with video segments. The details of the testing protocol vary depending on the subject matter to be tested and on any special properties of the subject 25.

FIG. 7 shows a particular audio segment 88 that comprises a 2.5 kHz tone that is played at various volumes 92 for different amounts of time. The audio segment 88 shown in FIG. 7 features first, second, and fourth tones that last for forty milliseconds each and a comparatively shorter third tone that only lasts for thirty milliseconds. The first, second, and third tones occur in succession. A 120-millisecond gap separates the third and fourth tones. A half-second rest separates the instances of the audio segment 88.

Having described the invention and a preferred embodiment thereof, what is claimed as new and secured by Letters Patent is:

1. An apparatus comprising a brain-training system for training neurons in a human's head to transition into a desired state, said brain-training system comprising a headset that comprises an electroencephalograph that extends along an arcuate path in a median plane of said human's head, wherein said electroencephalograph comprises plural dry electrodes, wherein said brain-training system further comprises an additional electrode that is configured to measure an additional physiological signal.

2. The apparatus of claim 1, wherein said additional electrode is configured for detecting an electromyographic signal.

3. The apparatus of claim 1, wherein one of said dry electrodes also functions as said additional electrode.

4. The apparatus of claim 1, wherein said additional electrode is a dry electrode.

5. The apparatus of claim 1, wherein said additional electrode is a wet electrode.

6. The apparatus of claim 1, wherein said additional electrode is disposed on the human's frontalis muscle.

7. The apparatus of claim 1, wherein one of said dry electrodes is used as said additional electrode.

8. The apparatus of claim 1, wherein said additional electrode is a dry electrode and wherein said dry electrode is extended to over said human's frontalis muscle.

9. The apparatus of claim 1, wherein said additional electrode is a dry electrode and wherein said dry electrode is extended to over said human's orbicularis oculi.

10. The apparatus of claim 1, wherein said headset comprises a reference sensor on an earcup thereof and said reference sensor is used as said additional electrode.

11. The apparatus of claim 1, wherein said headset comprises an earcup and wherein said additional electrode is on said earcup.

12. A method comprising exposing a subject to a test protocol while obtaining real-time electroencephalographic measurements and real-time measurements of an additional physiological signal and, based on said real-time measurements, determining a likelihood that said subject suffers from a disorder.

13. The method of claim 12, wherein said disorder is PTSD.

14. The method of claim 12, wherein said additional physiological signal is an electromyographic signal.

15. The method of claim 12, wherein said additional physiological signal is indicative of activity by said subject's orbicularis oculi.

16. The method of claim 12, wherein said additional physiological signal is indicative of activity by said subject's frontalis.

17. The method of claim 12, wherein said test protocol comprises plural audio segments separated by rests, wherein each of said audio segments comprises instances of a tone, said instances having different volumes and different durations.

18. The method of claim 12, wherein said test protocol comprises an audio segment.

19. The method of claim 12, wherein said test protocol comprises a video segment.

20. The method of claim 12, wherein said test protocol comprises an audio segment and a video segment.
